(19) United States
(12) Patent Application Publication
(10) Pub. No.: US 2025/0115005 A1
(43) Pub. Date: Apr. 10, 2025

(54) FUSION BONDING WAFER, TUBE FUSION BONDING DEVICE, AND TUBE FUSION BONDING METHOD

(71) Applicants: Terumo Kabushiki Kaisha, Tokyo (JP); Terumo BCT, Inc., Lakewood, CO (US)

(72) Inventors: Filip PAEPS, Leefdaal (BE); Maarten Roelandt, Kapellen (BE); Joachim Bortels, Sint-Truiden (BE); Lucas Vandekerckhove, Heverlee (BE)

(21) Appl. No.: 18/984,572

(22) Filed: Dec. 17, 2024

Related U.S. Application Data
(63) Continuation of application No. PCT/JP2022/036370, filed on Sep. 29, 2022.

(51) Int. Cl. B29C 65/30; B29C 65/22; B29C 65/78; B29L 23/00

(52) U.S. Cl. CPC B29C 65/30; B29C 65/22; B29C 65/7841; B29L 2023/22

(57) ABSTRACT

A fusion bonding wafer includes a first substrate formed in a flat plate shape, a second substrate joined to an inner surface of the first substrate, a heating element formed between the inner surface of the first substrate and an inner surface of the second substrate, and a temperature measurement hole that penetrates through the second substrate in a thickness direction thereof and exposes the inner surface of the first substrate.

### CROSS-REFERENCE TO RELATED APPLICATIONS

The present application is a continuation application of the International Patent Application No. PCT/JP2022/036370 filed on Sep. 29, 2022, which designated the U.S. The entire disclosure of the above-identified application is incorporated herein by reference.

### FIELD

The present disclosure relates to a fusion bonding wafer, a tube fusion bonding device, and a tube fusion bonding method configured to connect resin tubes by heating and fusion bonding them together.

### BACKGROUND

Tube fusion bonding devices may be used to aseptically connect resin tubes (hereinafter, referred to as tubes) such as tubes for medicinal use or the like. Tube fusion bonding devices may connect the tubes by melting and fusion bonding them together.

Tube fusion bonding devices may include a pair of clamps and a fusion bonding wafer. The pair of clamps may be configured to retain two tubes to be connected. The heated fusion bonding wafer may be inserted between the pair of clamps retaining the two tubes to intersect the tubes, and thereby cuts the two tubes. The pair of claims may be configured to move so that the tubes to be connected are placed face-to-face with each other with the fusion bonding wafer being interposed therebetween. When the fusion bonding wafer is pulled out, and the two tubes that face toward each other may be placed in direct contact to thereby fusion bond the two tubes. The fusion bonding wafer may be equipped with a heating element sandwiched between plate-shaped substrates. The heating element may be configured to heat the fusion bonding wafer to a predetermined temperature required for fusion bonding. It may be difficult to accurately detect a temperature when using the heated fusion bonding wafer.

### SUMMARY

In at least one example embodiment, the present disclosure provides a fusion bonding wafer configured to fusion bond two resin tubes together. The fusion bonding wafer may include a first substrate formed in a flat plate shape, a second substrate joined to an inner surface of the first substrate, a heating element disposed between the inner surface of the first substrate and an inner surface of the second substrate, and a temperature measurement hole that penetrates through the second substrate in a thickness direction thereof and is configured to expose the inner surface of the first substrate.

Also described herein is a tube fusion bonding device configured to cut two resin tubes by a fusion bonding wafer and to fusion bond the two resin tubes together. The tube fusion bonding device may include a pair of clamps configured to retain the two resin tubes, the fusion bonding wafer configured to be inserted between the pair of clamps, a temperature sensor configured to detect a temperature of the fusion bonding wafer, a clamp driving unit configured to cause at least one of the pair of clamps to be moved, a wafer driving unit configured to cause the fusion bonding wafer to be moved, and a controller configured to control operations of the clamp driving unit and the wafer driving unit. The fusion bonding wafer may include a first substrate formed in a flat plate shape, a second substrate joined to an inner surface of the first substrate, a heating element disposed between the inner surface of the first substrate and an inner surface of the second substrate, and a temperature measurement hole that penetrates through the second substrate in a thickness direction thereof and is configured to expose the inner surface of the first substrate, wherein the temperature sensor detects a temperature of the inner surface of the first substrate through the temperature measurement hole.

Also described herein is a tube fusion bonding method for fusion bonding two resin tubes together using a fusion bonding wafer. The fusion bonding wafer may include a first substrate formed in a flat plate shape, a second substrate joined to an inner surface of the first substrate, a heating element disposed between the inner surface of the first substrate and an inner surface of the second substrate, and a temperature measurement hole that penetrates through the second substrate in a thickness direction thereof and is configured to expose the inner surface of the first substrate. The fusion bonding method may include retaining two resin tubes by a pair of clamps, detecting a temperature of the fusion bonding wafer by a temperature sensor through the temperature measurement hole, pressing the fusion bonding wafer, by a wafer driving unit, into the two resin tubes retained by the pair of clamps and thereby cutting the two resin tubes, after a temperature of the inner surface of the first substrate as detected by the temperature sensor has reached a predetermined temperature, and fusion bonding the cut two resin tubes to each other.

In at least one example embodiment, a temperature of the fusion bonding wafer may be accurately detected via the fusion bonding wafer, the tube fusion bonding device, and the tube fusion bonding method described herein.

### BRIEF DESCRIPTION OF THE DRAWINGS

FIG. 1A is a perspective view of a tube fusion bonding device according to at least one example embodiment.

FIG. 1B is a perspective view of the tube fusion bonding device of FIG. 1A in which a fusion bonding wafer is made to project out therefrom according to at least one example embodiment.

FIG. 2A is a front view of the fusion bonding wafer shown in FIG. 1B according to at least one example embodiment.

FIG. 2B is a rear view of the fusion bonding wafer shown in FIG. 1B according to at least one example embodiment.

FIG. 3 is a plan view showing the fusion bonding wafer of FIG. 2 is in an unfolded state according to at least one example embodiment.

FIG. 4 is a cross-sectional view taken along line IV-IV of FIG. 2B according to at least one example embodiment.

FIG. 5 is an explanatory diagram showing a configuration of principal components of the tube fusion bonding device shown in FIG. 1 according to at least one example embodiment.

FIG. 6 is a flowchart showing a tube fusion bonding method in which the tube fusion bonding device of FIGS. 1A, 1B and 5 is used according to at least one example embodiment.

FIG. 7 is a plan view of another embodiment of a fusion boding wafer in an unfolded state according to at least one example embodiment.

FIG. 8 is a cross-sectional view of another fusion bonding wafer according to at least one example embodiment.

FIG. 9 is a plan view another embodiment of a fusion bonding wafer in an unfolded state according to at least one example embodiment.

### DETAILED DESCRIPTION

Hereinafter, a fusion bonding wafer (welding wafer) 18, a tube fusion bonding device (tubing welder) 10, and a tube fusion bonding method (tubing welding method) will be presented and described in detail below with reference to the accompanying drawings.

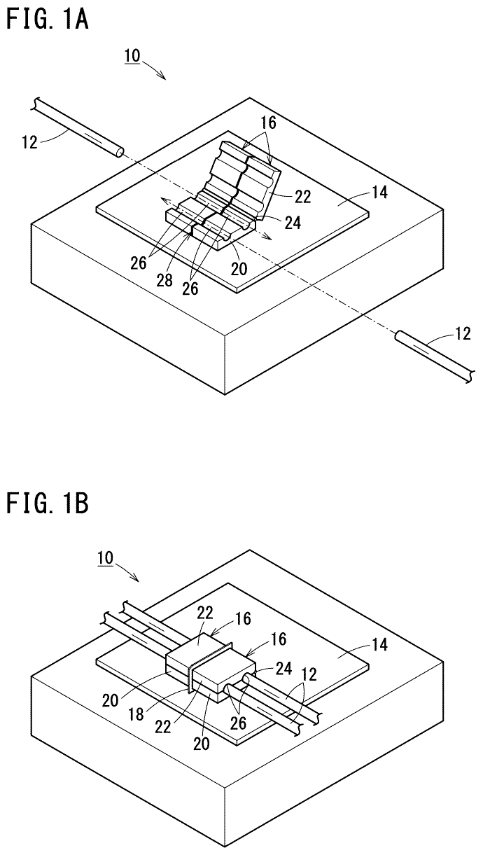

FIGS. 1A and 1B illustrate the tube fusion bonding device 10. The tube fusion bonding device may include a tube placement section 14 on which resin tubes 12 to be fusion bonded are placed. The tube placement section 14 may be provided with two clamps 16 that serve to retain the resin tubes 12, and the fusion bonding wafer 18.

In at least one example embodiment, each of the clamps 16 may be provided with a support member 20 and an arm member 22 which are vertically separated from each other.

The arm member 22 may be rotatably connected to the support member 20 via a hinge 24. Two grooves 26 configured to retain the resin tubes 12 in parallel may be provided on the upper surface of the support member 20. The two clamps 16 may be arranged alongside one another in parallel in a longitudinal direction. A narrow groove-shaped gap 28 may be disposed between the two clamps 16. In at least one example embodiment, the fusion bonding wafer 18 may be mounted in the gap 28 in an exchangeable manner.

Although not limited herein, the fusion bonding wafer 18 may be a disposable component that is replaced each time that fusion bonding is performed.

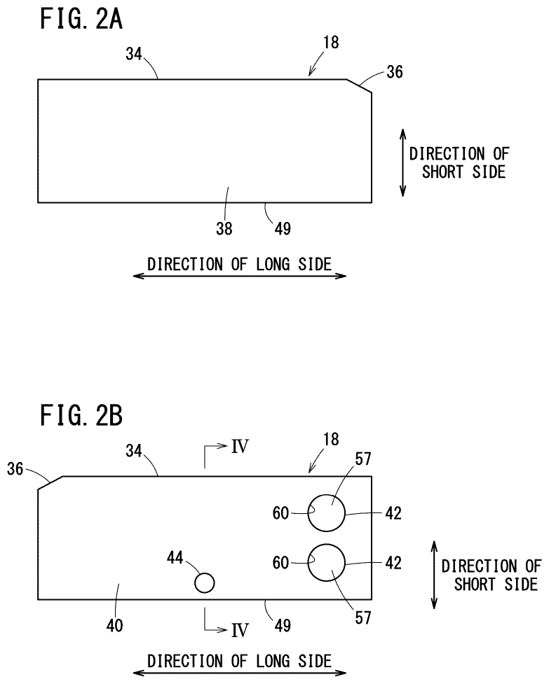

FIG. 2B illustrates an example embodiment of the fusion bonding wafer 18. The fusion bonding wafer 18 may be formed in a long rectangular shape, and one long side thereof serves as an incision side 34 that may be configured to cut into the resin tubes 12. In a corner adjacent to the incision side 34, a notch (cutout portion) 36 may be provided in order to indicate a position of the incision side 34. The notch 36 may also serve to identify the mounting direction of the fusion bonding wafer 18 in the tube fusion bonding device 10.

A front surface 38 of the fusion bonding wafer 18 may be a smooth plane. In contrast, as shown in FIG. 2B, a rear surface of the fusion bonding wafer 18 may include electrode portions 42 and a temperature measurement hole 44. The electrode portions 42 may be connected to connection terminals of the tube fusion bonding device 10. In at least one example embodiment, an electrical current for heating may be supplied to a wiring pattern 56, which is described in further detail below. The temperature measurement hole 44 may be used to detect the temperature in an interior of the fusion bonding wafer 18.

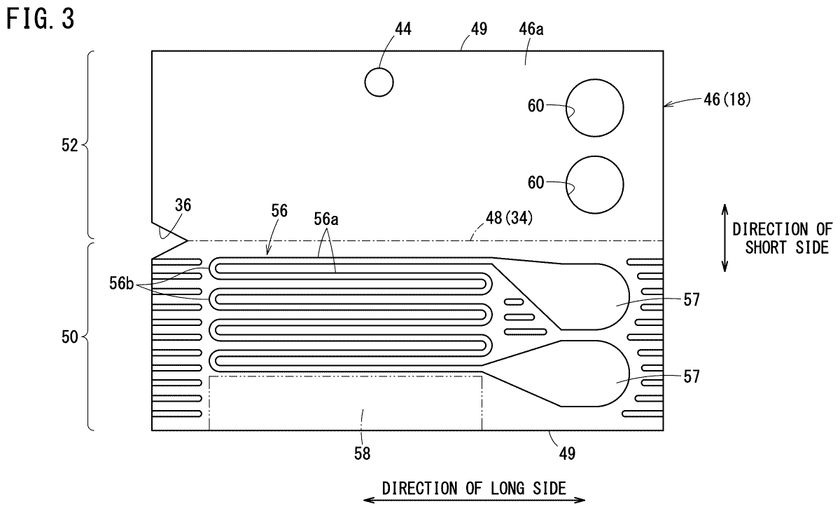

The fusion bonding wafer 18 may be formed by folding a thin metal plate 46 shown in FIG. 3. The thin metal plate 46, for example, may be a metal such as copper, a copper alloy, or stainless steel or the like, and may be formed in a rectangular shape wherein a dimension in a direction of the long side is on the order of 80 mm, and a dimension in a direction of the short side is on the order of 20 mm. A thickness of the thin metal plate 46, for example, may be on the order of 0.5 mm.

The thin metal plate 46 may be folded back on itself in an overlapping manner along a folding line 48 passing through the midpoints of the short sides to thereby form the fusion bonding wafer 18. As shown in FIG. 3, a portion on a lower side of the folding line 48 may include a first substrate 50, and a portion on an upper side of the folding line 48 may include a second substrate 52. More specifically, the first substrate 50 and the second substrate 52 may be integrally connected together through the folding line 48.

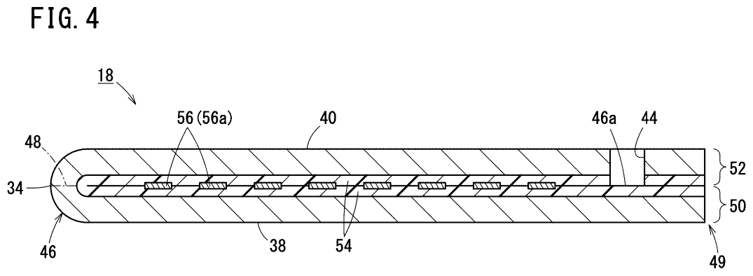

An inner surface 46a may not be visible on an outer side of the fusion bonding wafer 18. For example, as shown in FIG. 4, the inner surface 46a of the thin metal plate 46 may be covered with an insulating layer 54 made of a resin or the like. The insulating layer 54 may include a thermoplastic resin such as an acrylic resin or the like, and may function as an adhesive that joins the first substrate 50 and the second substrate 52 together by overlapping them in a heated state.

As shown in FIG. 3, in the inner surface 46a in an unfolded state, the wiring pattern 56 that constitutes the heating element is formed on the first substrate 50. The wiring pattern 56 may be formed by solidifying a powder of a conductive material such as silver or the like with a binder or the like such that the wiring pattern 56 is conductive. The wiring pattern 56 may be formed on the insulating layer 54. The wiring pattern 56 may include a plurality of linear portions 56a that extend in the direction of the long side, and curved portions 56b connecting the linear portions 56a. The wiring pattern 56 may be one meandering shape in which the linear portions 56a are connected by the curved portions 56b in the form of a U-turn. The shape of the wiring pattern 56 is not limited to a meandering shape, and various variations such as a plate-shaped pattern or the like can be adopted therefor.

In the first substrate 50, the wiring pattern 56 may be provided as a pattern that causes a uniform heat generation density to be generated in the vicinity of the incision side 34 (the folding line 48), which forms a side that cuts into the resin tubes 12 that serve as objects to be connected. The wiring pattern 56 may be separated away from an opposing side 49 that faces toward the folding line 48. A blank region 58 that does not include the wiring pattern 56 may be formed between the opposing side 49 and the wiring pattern 56. Further, connection pads 57 may be provided respectively at one end and the other end of the wiring pattern 56. The connection pads 57 may be wider than the linear portions 56a in at least one example embodiment. The connection pads 57 may be provided in close proximity to the short side on a side where the cutout portion 36 is not formed, and may be arranged alongside one another in the direction of the short side.

One pair of contact holes 60 and the temperature measurement hole 44 may be formed in portions of the second substrate 52. The contact holes 60 and the temperature measurement hole 44 are holes which are formed to penetrate through the second substrate 52 in the thickness direction, and for example, may be circular. When the second substrate 52 is overlapped on the first substrate 50, the contact holes may be formed in portions corresponding to the connection pads 57. As shown in FIG. 4, in a state in which the second substrate 52 is superimposed on the first substrate 50, the connection pads 57 may be exposed respectively at bottom parts of the contact holes 60. The electrode portions 42 may be constituted by the connection pads 57 and the contact holes 60.

As shown in FIG. 3, the temperature measurement hole 44 may be disposed at a site that avoids the wiring pattern 56 of the first substrate 50 and that does not interfere with the resin tubes 12 when the fusion bonding wafer 18 is pressed into the resin tubes 12. More specifically, the temperature measurement hole 44 may be provided in a portion of the second substrate 52 that corresponds to the blank region 58 in the vicinity of the opposing side 49 that faces toward the folding line 48 (the incision side 34).

As shown in FIG. 4, in a state in which the second substrate 52 is overlapped on the first substrate 50, the inner surface 46a (the insulating layer 54) of the first substrate 50 is exposed on a bottom part of the temperature measurement hole 44. To preventing errors in the temperature measurement result due to heat dissipation, the diameter of the temperature measurement hole 44 is preferably made as small as possible. For example, as shown in FIG. 3, the temperature measurement hole 44 is smaller than the contact holes 60. Although not particularly limited to this feature, the diameter of the temperature measurement hole 44 can be on the order of 1 to 2 mm.

In at least one example embodiment, the inner surface 46a of the thin metal plate 46 may be coated with an acrylic resin or the like to thereby form the insulating layer 54 and thereafter the thin metal plate is formed into the rectangular shape shown in FIG. 3, by means of press molding to form the fusion bonding wafer 18. Next, the contact holes 60 and the temperature measurement hole 44 may be punched out and formed. Thereafter, the wiring pattern 56 may be formed on the insulating layer 54 by coating thereon an ink containing a conductive material such as silver or the like by a screen printing method or the like. Thereafter, the thin metal plate 46 is folded along the folding line 48, the second substrate 52 may be overlaid on the first substrate 50, and by heating and pressing them, the first substrate 50 and the second substrate 52 may be joined together via the insulating layer 54, thereby completing the fusion bonding wafer 18 having the cross-sectional structure shown in FIG. 4.

The fusion bonding wafer 18 may be used with the tube fusion bonding device 10 shown in FIG. 1B.

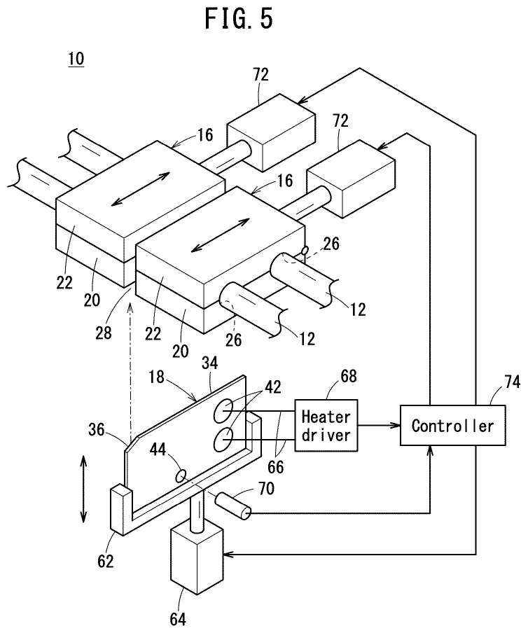

As shown in FIG. 5, the tube fusion bonding device 10 may be equipped with a holder 62. In at least one example embodiment, the holder 62 may retain the fusion bonding wafer 18 in a detachable manner. The holder 62 may be supported by a wafer driving unit 64 that is configured to cause the holder 62 to move in a vertical (up-down) direction (a direction across the resin tubes 12). Electrode terminals 66 that are in contact with the electrode portions 42 of the fusion bonding wafer 18 may be provided in the vicinity of the holder 62. The wiring pattern 56 of the fusion bonding wafer 18 may be connected to a heater driver 68 via the electrode terminals 66.

Further, the tube fusion bonding device 10 may include a temperature sensor 70, that may be disposed at a position face-to-face with the temperature measurement hole 44 of the fusion bonding wafer 18. In at least one example embodiment, the temperature sensor 70 may be an infrared radiation thermometer that may be configured to detect the temperature of the inner surface 46a that is exposed through the temperature measurement hole 44 in a non-contact manner. The temperature sensor 70 is not limited to being the infrared radiation thermometer, and may be a contact sensor in which a temperature measurement probe is placed in contact with the inner surface 46a.

The pair of clamps 16 may be disposed on both sides of the holder 62, and the clamps 16 may be supported by clamp driving units 72. The clamp driving units 72 may be configured to move the clamps 16 in a direction perpendicular to the direction in which the wafer driving unit 64 moves the wafer (i.e., in the longitudinal direction of the clamps 16).

In the tube fusion bonding device 10, the wafer driving unit 64, the heater driver 68, the temperature sensor 70, and the clamp driving units 72 may be connected to a controller 74. The tube fusion bonding device 10 may be configured to carry out a fusion bonding operation of the resin tubes 12 under the control of a control operation of the controller 74.

A tube fusion bonding method may involve both the fusion bonding wafer 18 and the tube fusion bonding device 10.

In at least one example embodiment, prior to fusion bonding the resin tubes 12, a user may mount the fusion bonding wafer 18 on the tube fusion bonding device 10 shown in FIGS. 1A and 1B. Further, the two resin tubes 12 that serve as objects to be connected are set in parallel on the clamps 16. Thereafter, when the user presses a start button of the tube fusion bonding device 10, a fusion bonding operation of the tube fusion bonding device 10 may be initiated.

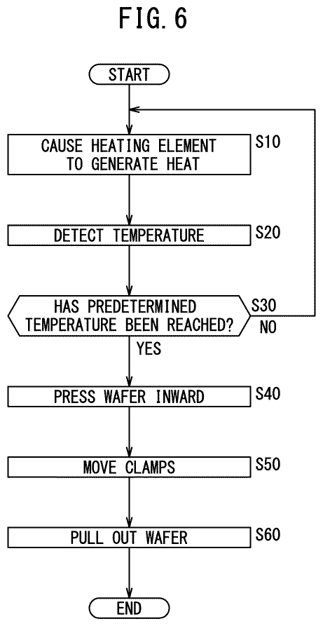

FIG. 6 illustrates a flow chart of the tube fusion bonding method. The tube fusion bonding method may begin in step S10 where the tube fusion bonding device 10 may cause the heating element (the wiring pattern 56) of the fusion bonding wafer 18 to generate heat. More specifically, the tube fusion bonding device 10 may supply an electrical current to the electrode portions 42 of the fusion bonding wafer 18 via the heater driver 68, and may heat the fusion bonding wafer 18 by resistance heat generation of the wiring pattern 56.

Next, in step S20, the tube fusion bonding device 10 may detect a temperature of the fusion bonding wafer 18. In at least one example embodiment, the temperature sensor 70 may detect the temperature of the inner surface 46a of the fusion bonding wafer 18 through the temperature measurement hole 44 of the fusion bonding wafer 18. The heat generated in the wiring pattern 56 of the fusion bonding wafer 18 may be transmitted to the surfaces of the first substrate 50 and the second substrate 52 due to thermal conduction which may heat surfaces of the first substrate 50 and the second substrate 52. Since the surfaces of the first substrate 50 and the second substrate 52 dissipate heat to the external air, the temperature tends to decrease as the distance in a planar direction from the wiring pattern 56 increases. Thus, the temperature may vary significantly within the surface. In order to measure the temperature in the vicinity of the incision side 34 that cuts into the resin tubes 12, it may be preferable to dispose the temperature sensor 70 in the vicinity thereof. However, this placement may interfere with the resin tubes 12. Thus, in order to prevent interference with the resin tubes 12, the temperature of the fusion bonding wafer 18 may be detected at a site that is distanced from the incision side 34.

In at least one example embodiment, the temperature sensor 70 of the present embodiment may detect the temperature in the interior of the fusion bonding wafer 18 through the temperature measurement hole 44 of the fusion bonding wafer 18. Because, the influence of release of heat to the exterior air is small at the inner surface 46a of the fusion bonding wafer 18, even at a site separated away from the incision side 34, the temperature of the inner surface 46a of the fusion bonding wafer 18 may be roughly the same as the temperature in the vicinity of the incision side 34. Therefore, the temperature sensor 70 may accurately measure the temperature in the vicinity of the incision side 34.

The temperature detected by the temperature sensor 70 may be input to the controller 74, and in step S30, the controller 74 may determine whether a predetermined temperature has been reached. In at least one example embodiment, the controller 74 may determine whether or not the temperature of the fusion bonding wafer 18 has reached a predetermined temperature that is greater than or equal to the melting point of the resin tubes 12. In at least one example embodiment, the predetermined temperature may be about 280 degrees centigrade. However, example embodiments are not limited herein.

If the controller 74 determines that the temperature has not reached the predetermined temperature (NO) at step S30, the process returns to step S20, and measurement of the temperature is continued. If the controller 74 determines that the fusion bonding wafer 18 has reached the predetermined temperature (YES) at step S30, the process transitions to step S40.

In step S40, the tube fusion bonding device 10 may drive the wafer driving unit 64, move the fusion bonding wafer 18 upward, and press the fusion bonding wafer into the two resin tubes 12 to cut across the two resin tubes 12. Thus, in at least one example embodiment, the fusion bonding wafer 18 may cut into the resin tubes 12 from the incision side 34 and cut through the resin tubes 12 while in close contact with the resin tubes 12.

Next, in step S50, the tube fusion bonding device 10 may drive the clamp driving units 72 to cause the two resin tubes 12, which are fusion bonded target, to be placed at positions face-to-face with each other. Consequently, one of the resin tubes 12 and the other of the resin tubes 12 that serve as objects to be connected may be arranged face-to-face with each other with the fusion bonding wafer 18 being interposed therebetween. Because the ends of the resin tubes 12 are in close contact with the heated surfaces of the fusion bonding wafer 18, the ends and the interior of the resin tubes 12 are maintained in a sterile condition.

Next, in step S60, the tube fusion bonding device 10 may drive the wafer driving unit 64, causes the fusion bonding wafer 18 to move downward which removes the fusion bonding wafer 18 from between the pair of resin tubes 12. Thus, the melted end of one of the resin tubes 12 and the melted end of the other of the resin tubes 12 are brought into face-to-face contact with each other, and are fusion bonded together. In at least one example embodiment, the resin tubes 12 are fusion bonded while the interiors of the resin tubes 12 are maintained in a sterile condition.

In at least one example embodiment, the fusion bonding wafer 18 is configured to fusion bond the two resin tubes 12 together and may include the first substrate 50 which is formed in a flat plate shape, the second substrate 52 joined to the inner surface 46a of the first substrate 50, the heating element (e.g., the wiring pattern 56) disposed between the inner surface 46a of the first substrate 50 and the inner surface 46a of the second substrate 52, and the temperature measurement hole 44 that penetrates through the second substrate 52 in the thickness direction and is configured to expose the inner surface 46a of the first substrate 50.

Because the temperature of the inner surface 46a of the interior of the fusion bonding wafer 18 may be detected, even in the case that the position of the temperature measurement hole 44 is located at a position separated away from the heating element, the temperature of the surface of the fusion bonding wafer 18 in the vicinity of the heating element may be accurately measured.

In at least one example embodiment, the temperature measurement hole 44 may be disposed at a site that avoids the heating element. Thus, measurement of the temperature may be carried out at a position where the influence of heat generated by the heating element is avoided which may minimize an error in measuring the temperature of the fusion bonding wafer 18.

In at least one example embodiment, the first substrate 50 and the second substrate 52 may each include the incision side 34 that cuts into the resin tubes 12 that serve as objects to be fusion bonded and the opposing side 49 formed on an opposite side from the incision side 34. The temperature measurement hole 44 may be disposed in the vicinity of the opposing side 49 such that the temperature measurement hole 44 is provided at a position that does not interfere with the resin tubes 12.

In at least one example embodiment, the first substrate 50 and the second substrate 52 may be formed by being connected together integrally. In particular, the second substrate 52 may be folded back toward the inner surface 46a of the first substrate 50 at the incision side 34. Because the first substrate 50 and the second substrate 52 may be formed simultaneously by press working, mass productivity thereof is superior, and manufacturing costs may be reduced.

In at least one example embodiment, the inner surface 46a of the first substrate 50 and the inner surface 46a of the second substrate 52 may each be covered with the insulating layer 54, and the heating element may be made up from the wiring pattern 56 that is formed on the insulating layer 54 of either one of the first substrate 50 or the second substrate 52. Because the wiring pattern 56 may be formed by a printing method, the production cost of the fusion bonding wafer 18 may be reduced.

In at least one example embodiment, the wiring pattern 56 may include the connection pads 57 on one end and another end thereof, and the contact holes 60 by which the connection pads 57 are exposed may be formed in one or both of the first substrate 50 and the second substrate 52. Thus, electrical current may flow to the wiring pattern 56 through the contact holes 60.

In at least one example embodiment, the heating element (the wiring pattern 56) may be formed on the insulating layer 54 of the first substrate 50.

In at least one example embodiment, the tube fusion bonding device 10 may be configured to cut the two resin tubes 12 by the fusion bonding wafer 18 and fusion bond the two resin tubes 12 together. The tube fusion bonding device 10 may include the pair of clamps 16 that retain the two resin tubes 12, the fusion bonding wafer 18 configured to be inserted between the pair of clamps 16, the temperature sensor 70 configured to detect the temperature of the fusion bonding wafer 18, the clamp driving unit 72 configured to cause at least one of the pair of clamps 16 to be moved, the wafer driving unit 64 configured to cause the fusion bonding wafer 18 to be moved, and the controller 74 configured to control operations of the clamp driving unit 72 and the wafer driving unit 64. The fusion bonding wafer 18 may include the first substrate 50 formed in a flat plate shape, the second substrate 52 joined to the inner surface 46a of the first substrate 50, the heating element disposed between the inner surface 46a of the first substrate 50 and the inner surface 46a of the second substrate 52, and the temperature measurement hole 44 that penetrates through the second substrate 52 in the thickness direction and exposes the inner surface 46a of the first substrate 50. The temperature sensor 70 may detect the temperature of the inner surface 46a of the first substrate 50 through the temperature measurement hole 44.

According to the above-described tube fusion bonding device 10, the temperature of the fusion bonding wafer 18 may be accurately measured, even when the temperature sensor 70 is disposed at a position that does not interfere with the resin tubes 12.

In at least one example embodiment, the controller 74 may control operation of the wafer driving unit 64 to press the fusion bonding wafer 18 into the two resin tubes 12 retained by the pair of clamps 16 after the temperature of the inner surface 46a of the first substrate 50 as detected by the temperature sensor 70 has reached the predetermined temperature. In at least one example embodiment, a rise in temperature of the fusion bonding wafer 18 may be detected quickly which may reduce a waiting time for raising the temperature of the fusion bonding wafer 18. Thus, the two resin tubes 12 may be fusion bonded together more quickly.

In at least one example embodiment, the temperature sensor 70 may be a non-contact type of temperature sensor. Thus, deterioration of the temperature sensor 70 may be prevented, even when replacement of the fusion bonding wafer 18 is frequently performed.

The tube fusion bonding method described herein may be a tube fusion bonding method for fusion bonding the two resin tubes 12 together using the fusion bonding wafer 18, wherein the fusion bonding wafer 18 includes the first substrate 50 formed in a flat plate shape, the second substrate 52 joined to the inner surface 46a of the first substrate 50, the heating element disposed between the inner surface 46a of the first substrate 50 and the inner surface 46a of the second substrate 52, and the temperature measurement hole 44 that penetrates through the second substrate 52 in the thickness direction and exposes the inner surface 46a of the first substrate 50. The fusion bonding method may include the steps of retaining the two resin tubes 12 by the pair of clamps 16, detecting the temperature of the fusion bonding wafer 18 by the temperature sensor 70 through the temperature measurement hole 44, pressing the fusion bonding wafer 18, by the wafer driving unit 64, into the two resin tubes 12 retained by the pair of clamps 16 and thereby cutting the two resin tubes 12 after the temperature of the inner surface 46a of the first substrate 50 as detected by the temperature sensor 70 has reached the predetermined temperature, and fusion bonding the cut two resin tubes to each other.

In at least one example embodiment, fusion bonding of the resin tubes 12 may be carried out reliably and quickly because the fusion bonding wafer 18 is pressed into the resin tubes 12 after the temperature sensor 70 has detected an accurate temperature of the fusion bonding wafer 18.

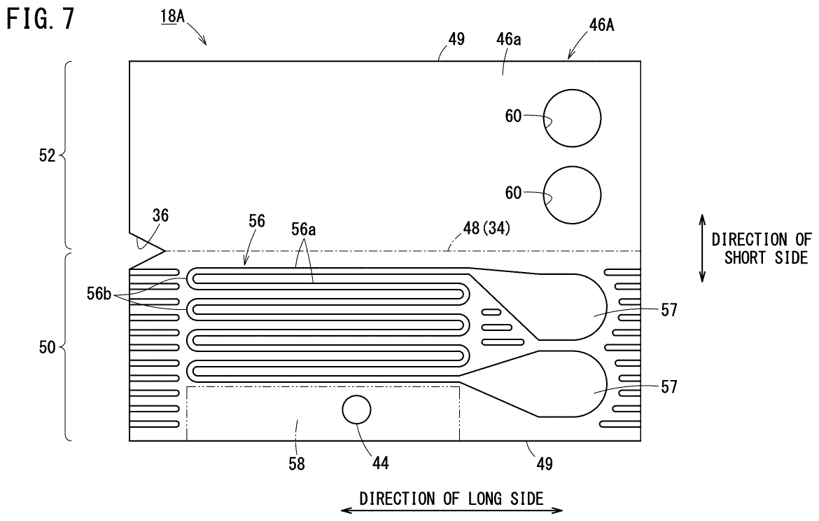

FIG. 7 illustrates another embodiment of a thin metal plate 46A (fusion bonding wafer 18A). In at least one example embodiment, the temperature measurement hole 44 may be provided on the side of the first substrate 50 and not on the side of the second substrate 52. When the first substrate 50 and the second substrate 52 are overlapped, the inner surface 46a on the side of the second substrate 52 may be exposed through the temperature measurement hole 44. Since the inner surface 46a of the first substrate 50 and the inner surface 46a of the second substrate 52 are located in close proximity to each other, the inner surface 46a of the second substrate 52 may exhibit similar thermal properties to those of the inner surface 46a of the first substrate 50 which may enable an accurate temperature measurement to be carried out.

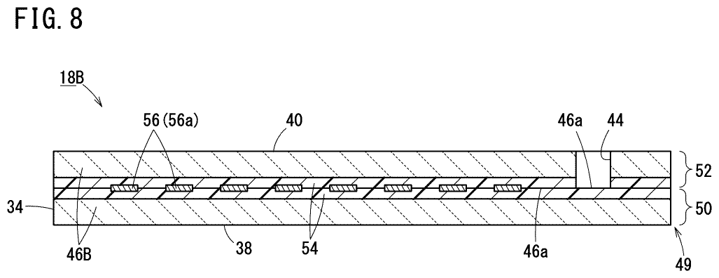

FIG. 8 illustrates a fusion bonding wafer 18B where the first substrate 50 and the second substrate 52 are made up from separate members. In particular, the first substrate 50 and the second substrate 52 may be separated at the incision side 34. The first substrate 50 and the second substrate 52 of the fusion bonding wafer 18B may be constituted by a thin metal plate 46B and the insulating layer 54 provided on the side of the inner surface 46a thereof. However example embodiments are not limited herein. In at least one example embodiment, instead of the thin metal plate 46B, for example, a material that is difficult to bend such as a ceramic substrate or the like may be used. Thus, a wide choice of materials may be used which may allow less expensive materials to be utilized.

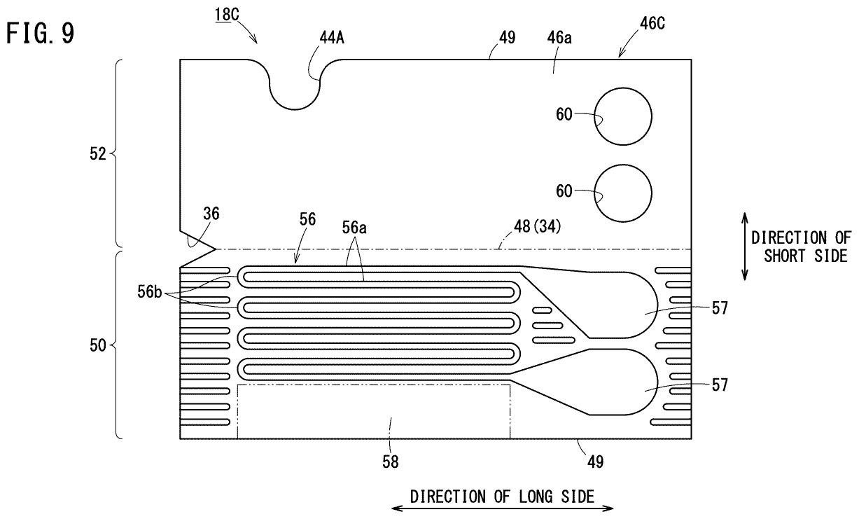

FIG. 9 illustrates a thin metal plate 46C where a temperature measurement hole 44A provided in the second substrate 52 intersects the opposing side 49. The temperature measurement hole 44A may be formed as a notch (cutout portion) that is opened on the side of the opposing side 49. The thin metal plate 46C may provide similar effects as the fusion bonding wafer 18 described with reference to FIGS. 2 through 4.

Although several example embodiments of the present invention have been described above, the present disclosure is not limited to the above-described embodiments. Various modifications could be adopted therein without departing from the essence and gist of the present disclosure.

1. A fusion bonding wafer configured to fusion bond two resin tubes together, the fusion bonding wafer comprising:
a first substrate formed in a flat plate shape;
a second substrate joined to an inner surface of the first substrate;
a heating element disposed between the inner surface of the first substrate and an inner surface of the second substrate; and
a temperature measurement hole that penetrates through the second substrate in a thickness direction thereof and is configured to expose the inner surface of the first substrate.

2. The fusion bonding wafer of claim 1, wherein the temperature measurement hole is disposed at a location that avoids the heating element.

3. The fusion bonding wafer of claim 1, wherein
the first substrate and the second substrate each include an incision side configured to cut into the resin tubes and an opposing side opposite from the incision side; and
the temperature measurement hole is disposed in a vicinity of the opposing side.

4. The fusion bonding wafer of claim 3, wherein a portion of the temperature measurement hole intersects the opposing side.

5. The fusion bonding wafer of claim 3, wherein the first substrate and the second substrate are formed by being connected together integrally, and the second substrate is folded back toward the inner surface of the first substrate at the incision side.

6. The fusion bonding wafer of claim 1, wherein the inner surface of the first substrate and the inner surface of the second substrate are each covered with an insulating layer.

7. The fusion bonding wafer of claim 6, wherein the temperature measurement hole is provided in the first substrate, and the heating element is formed on the insulating layer of the first substrate.

8. The fusion bonding wafer of claim 1, wherein the heating element includes a wiring pattern formed on the insulating layer of the first substrate or the second substrate.

9. The fusion bonding wafer of claim 8, wherein
the wiring pattern includes a first connection pad on a first end of the wiring pattern and a second connection pad on a second end of the wiring pattern, and
a first contact hole configured to expose the first connection pad is exposed is formed in one or both of the first substrate and the second substrate, and
a second contact hole configured to expose the second connection pad is exposed is formed in one or both of the first substrate and the second substrate.

10. A tube fusion bonding device configured to cut two resin tubes and fusion bond the two resin tubes together, the tube fusion bonding device comprising:
a pair of clamps configured to retain the two resin tubes;
a fusion bonding wafer configured to be inserted between the pair of clamps, the fusion bonding wafer including
a first substrate formed in a flat plate shape,
a second substrate joined to an inner surface of the first substrate,
a heating element disposed between the inner surface of the first substrate and an inner surface of the second substrate, and
a temperature measurement hole that penetrates through the second substrate in a thickness direction thereof and is configured to expose the inner surface of the first substrate;
a temperature sensor configured to detect a temperature of the fusion bonding wafer;
a clamp driving unit configured to cause at least one of the pair of clamps to be moved;
a wafer driving unit configured to cause the fusion bonding wafer to be moved; and
a controller configured to control operations of the clamp driving unit and the wafer driving unit,
wherein the temperature sensor is configured to detect a temperature of the inner surface of the first substrate through the temperature measurement hole.

11. The tube fusion bonding device of claim 10, wherein the controller is configured to control operation of the wafer driving unit to press the fusion bonding wafer into the two resin tubes retained by the pair of clamps after the temperature of the inner surface of the first substrate has reached a predetermined temperature.

12. The tube fusion bonding device of claim 10, wherein the temperature sensor is a non-contact temperature sensor.

13. The tube fusion bonding device of claim 10, wherein the temperature measurement hole is disposed at a location that avoids the heating element.

14. The tube fusion bonding device of claim 10, wherein
the first substrate and the second substrate each include an incision side configured to cut into the resin tubes and an opposing side opposite from the incision side; and
the temperature measurement hole is disposed in a vicinity of the opposing side.

15. The tube fusion bonding device of claim 14, wherein a portion of the temperature measurement hole intersects the opposing side.

16. The tube fusion bonding device of claim 14, wherein the first substrate and the second substrate are formed by being connected together integrally, and the second substrate is folded back toward the inner surface of the first substrate at the incision side.

17. The tube fusion bonding device of claim 10, wherein the inner surface of the first substrate and the inner surface of the second substrate are each covered with an insulating layer and wherein the temperature measurement hole is provided in the first substrate, and the heating element is formed on the insulating layer of the first substrate.

18. The tube fusion bonding device of claim 10, wherein the heating element includes a wiring pattern formed on the insulating layer of the first substrate or the second substrate.

19. The tube fusion bonding device of claim 18, wherein
the wiring pattern includes a first connection pad on a first end of the wiring pattern and a second connection pad on a second end of the wiring pattern, and
a first contact hole configured to expose the first connection pad is exposed is formed in one or both of the first substrate and the second substrate, and
a second contact hole configured to expose the second connection pad is exposed is formed in one or both of the first substrate and the second substrate.

20. A tube fusion bonding method for fusion bonding two resin tubes together using a fusion bonding wafer, the method comprising:
retaining two resin tubes by a pair of clamps;
detecting a temperature of the fusion bonding wafer by a temperature sensor through the temperature measurement hole;
pressing the fusion bonding wafer, by a wafer driving unit, into the two resin tubes retained by the pair of clamps to cut the two resin tubes in response to a temperature of the inner surface of the first substrate reaching a predetermined temperature; and
fusion bonding the cut two resin tubes to each other,
wherein the fusion bonding wafer includes
a first substrate formed in a flat plate shape,
a second substrate joined to an inner surface of the first substrate,
a heating element disposed between the inner surface of the first substrate and an inner surface of the second substrate, and
a temperature measurement hole that penetrates through the second substrate in a thickness direction thereof and is configured to expose the inner surface of the first substrate.
